(19) United States
(12) Patent Application Publication
(10) Pub. No.: US 2005/0096545 A1
(43) Pub. Date: May 5, 2005

(54) Methods and apparatus for transducer probe

(76) Inventors: Bruno Hans Haider, Ballston Lake, NY (US); Robert Gideon Wodnicki, Niskayuna, NY (US)

Correspondence Address: GENERAL ELECTRIC COMPANY (PCPI);C/O FLETCHER YODER, P. O. BOX 692289, HOUSTON, TX, 77269-2289, US

(21) Appl. No.: 10/697,518

(22) Filed: Oct. 30, 2003

(51) Int. Cl. A61B 8/06

(52) U.S. Cl. 600/447; 600/459

(57) ABSTRACT

A probe having a plurality of transducers also has a plurality of pulsers within the probe that are responsive to one or more transmit timing signals received from an external system to transmit pulses to the plurality of transducers. The external system may be, but need not be, an imaging system, and the transducers may be, but need not be, ultrasound transducers.

### BACKGROUND OF THE INVENTION

This invention relates generally to transducer probes, and more generally to methods and apparatus for efficiently operating a probe having a large number of transducer elements.

Conventional medical ultrasound imaging creates two dimensional, cross-sectional images using one dimensional linear or phased array transducers. These transducers are built with approximately 100 to 200 elements arranged in a linear fashion. The transducer elements (also referred to more simply as “transducers”) are connected to high voltage pulsers in the system. The pulsers send waveforms to the transducer elements, which in turn convert the electrical waveforms into acoustic waves. By properly controlling the waveforms, a focused sound beam is created. The signal level of the electrical waveforms can be several hundred volts in order to generate the desired level of acoustic energy. Connecting a few hundred transducer elements to the system is technically feasible with current technology.

Two dimensional transducer arrays are required for three dimensional imaging. These types of transducers employ several thousand elements. For proper beamforming, each one of these elements must be connected to a beamforming channel. Connecting several thousand elements to a pulser in the system is technically not feasible in that a cable bundle of coax or other wire comprising a sufficient number of conductors for several thousand elements would be too thick and heavy to be ergonomically viable. Also, a cable that would connect the system pulser to the transducer element would present a very large capacitance load compared to the impedance of the two-dimensional array element. Therefore, a majority of the pulser's current would be drawn into the cable capacitance while only a small fraction of the current would remain for the transducer element. As a result, only a small fraction of the energy supplied by the pulser would be converted to acoustic waves. Consequently, much more power would have to be supplied by the pulser circuitry than would be required from a linear array. This additional power requirement might be tolerable for a full-size clinical ultrasound scanner. However, it would be prohibitive for a portable system, which would not be able to supply sufficient cooling for the pulsers. In addition, the portable system would suffer drastically reduced battery life.

### BRIEF DESCRIPTION OF THE INVENTION

Some configurations of the present invention therefore provide a probe having a plurality of transducers. The probe also has a plurality of pulsers within the probe that are responsive to one or more transmit timing signals received from an external system to transmit pulses to the plurality of transducers.

Also, some configurations of the present invention provide a probe having a plurality of transducers. The probe also includes a transmit timing circuit in the probe handle that is responsive to one or more control signals received from an external system to generate timing signals and a plurality of pulsers within the probe that are responsive to the timing signals to generate high voltage pulses. The probe also includes a plurality of transducers that are responsive to the high voltage pulses.

Still other configurations of the present invention provide a probe that includes a plurality of transducers. The probe also includes an array of pulsers, wherein each transducer element is responsive to pulses from a dedicated pulser. The probe also contains a low voltage multiplexer that is responsive to a control signal from an external system and which is configured to distribute signals to the array of pulsers. The pulsers are responsive to the signals from the multiplexer to generate pulses to the transducers.

Yet other configurations of the present invention provide a probe having a plurality of transducers. The probe also includes an array of pulsers, wherein each transducer is responsive to pulses from a dedicated pulser. Also provided in the probe is an array of transmit timing circuits within the probe that are responsive to one or more control signals received from an external system to generate timing signals. The timing circuits include a memory, and the pulsers are responsive to the timing signals from the array of timing circuits to generate pulses to the transducers.

Still other configurations of the present invention provide a probe that includes a plurality of transducers and a plurality of pursers within the probe. The pulsers are responsive to one or more timing signals to transmit pulses to the plurality of transducers. A transmit timing is included within the probe. The transmit timing circuit is configured to generate the one or more timing signals. A pulse timing and control circuit is also included in the probe to control the transmit timing circuit.

Moreover, still other configurations of the present invention provide a method for operating a transducer probe. The method includes generating one or more signals in an external system, controlling a plurality of pulsers in a probe utilizing the signals from the external system, and operating a plurality of transducers utilizing signals from the plurality of pulsers.

Yet other configurations of the present invention provide a method for operating a transducer probe. These configurations include generating one or more signals in the transducer probe, controlling a plurality of pulsers in the probe utilizing the one or more signal generated in the transducer probe, and operating a plurality of transducers utilizing signals from the plurality of pulsers.

In some configurations of the present invention, the probe is an ultrasound probe and the transducers are ultrasound transducers, but the present invention is not limited to configurations of ultrasound probes or of probes that utilize ultrasound transducers.

It will thus be observed that configurations of the present invention provide the ability to transmit with very small elements and with larger numbers of elements than the number of available system channels. Also, configurations of the present invention provide these advantages without the need to provide large numbers of cables between an imaging system and a probe handle, and without presenting an excessively large capacitive load between transducers and pulsers. Moreover, these benefits accrue without the need for excessive power that would otherwise be required of other portable probe configurations.

### BRIEF DESCRIPTION OF THE DRAWINGS

FIG. 1 is a block diagram representing various configurations of the present invention in which a plurality of pulsers in a handle of a transducer probe are controlled by digital timing signals received from an imaging system.

FIG. 2 is a block diagram representing various configurations of the present invention in which a plurality of pulsers in a handle of a transducer probe are controlled by analog timing signals received from an imaging system.

FIG. 3 is a block diagram representing various configurations of the present invention in which a plurality of pulsers in a handle of a transducer probe are directly controlled by timing signals received from an imaging system.

FIG. 4 is a block diagram representing various configurations of the present invention in which an array of pulsers in a handle of a transducer probe are controlled by a multiplexer receiving timing signals from an imaging system.

FIG. 5 is a block diagram representing various configurations of the present invention in which an array of pulsers in a handle of a transducer probe are controlled by a timing circuit contained within the handle.

FIG. 6 is a block diagram representing various configurations of the present invention in which an array of pulsers in a handle of a transducer probe are controlled by a timing circuit array that is also in the transducer probe.

It will be understood that in the Figures, only a representative portion of replicated circuitry is shown. In some instances, replication clearly and specifically implied in the description (e.g., “every transducer element has a dedicated low current HV pulse transmitted”) is not explicitly indicated in the Figures due to lack of space. Moreover, imaging processing subcomponents and displays that are not necessary to convey an understanding of the present invention are not shown in the Figures.

### DETAILED DESCRIPTION OF THE INVENTION

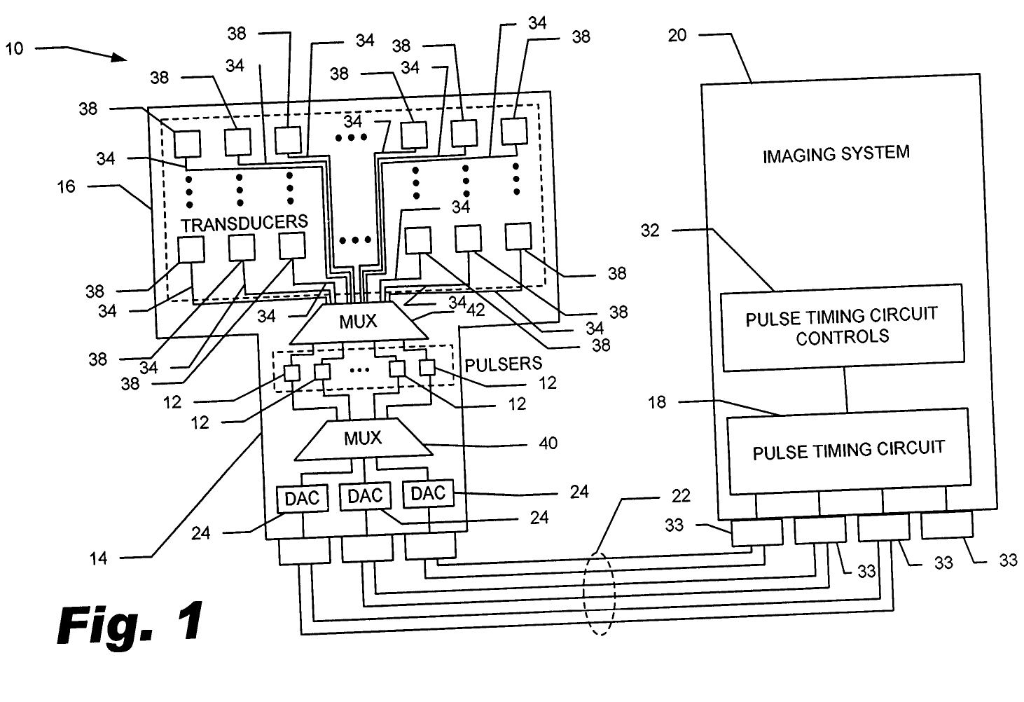

In some configurations 10 of the present invention and referring to FIG. 1, high voltage (HV) pulse transmitters 12 (also referred to as “pulsers”) are disposed in a handle 14 of an ultrasound probe 16. In various configurations, pulsers 12 comprise unipolar, bipolar, or multi-level pulsers, or a combination thereof. Placing pulsers 12 in handle 14 advantageously permits pulse timing circuitry 18 to be located either in imaging system 20, as shown in FIG. 1, or in probe handle 14. (To reduce the complexity of the Figures, components and circuitry associated with the detection and receiving of reflected ultrasound signals in probe 16, transmitting received data from probe 16 to imaging system 20 and processing the data to generate an image are omitted in the Figures. The omitted components and circuitry are conventional and are not part of the present invention.)

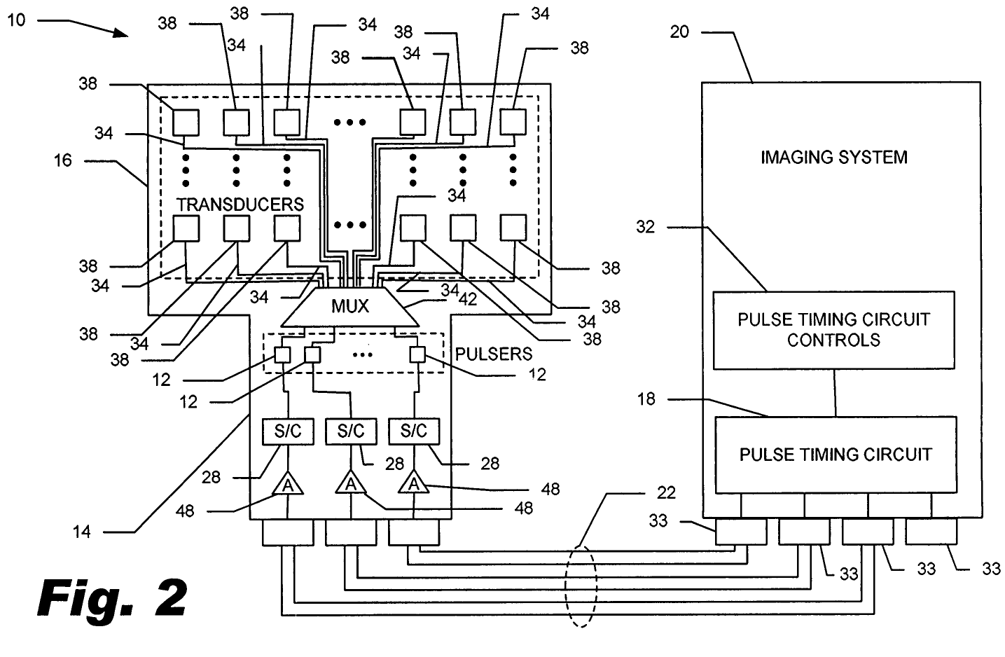

In some configurations in which pulse timing circuit 18 is located in imaging system 20 and pulsers 12 are located in probe handle 14, timing information generated by imaging system 20 is transmitted in a low voltage format over one or more probe cables 22 to probe handle 24. In configurations using a digital timing format, one or more digital to analog converters (DACs) 24 are located in probe handle 14 to convert timing signals to an analog format for driving high voltage (HV) pulse transmitter circuits 12. In some configurations, pulsers 12 are bipolar or unipolar pulsers, or a combination thereof, and circuitry 24 (for example, digital circuitry instead of DACs shown in FIG. 1) is provided to convert control and timing signals from imaging system 20 to low voltage signals that operate pulsers 12. In configurations in which the timing format is analog, and referring to FIG. 2, signal conditioners (S/C) 28 and/or amplifiers (A) 48 are used to convert the low voltage analog timing signals into driving signals to control HV pulse transmitter circuits 12. Regardless of the timing format, some configurations of imaging system 20 are configured to allow the utilization of the timing signals to specify unipolar, bipolar, or multilevel pulses. Also, some configurations of imaging system 20 are configured to allow the utilization of the timing signals to specify varying time delay, pulse width, and/or pulse number. Multiple pulses of varying timing can be transmitted during each imaging time in some configurations. Controls 32 may be provided for such selection, or the selection may be made via an electronic handshake, via separate cable connectors 33, or by other suitable means.

Multiple, simultaneously operating HV pulse channels 34 are provided in some configurations for focused ultrasound transmit beam formation. Parameters of the pulse train in each channel 34 are varied to achieve focused ultrasound transmission. Pulse timing circuit 16 generates multiple low voltage timing signals that are propagated on a plurality of coaxial cables 22 from imaging system 20 to probe 16 in which pulsers 12 are located. When the timing signals reach probe handle 14, they are routed to individual pulsers 12, and from pulsers 12 they are routed to individual transducer elements 38. Multiplexers 40 and 42 are reprogrammed before each transmit operation to provide a many-to-many mapping from the low voltage timing signal to pulsers 12, and from pulsers 12 to transducers 38, respectively. Not all configurations include both multiplexers 40 and 42, and some configurations omit both multiplexers 40 and 42. Some configurations omitting either or both multiplexers compensate for the omission by including a larger number of pulsers 12 to control the same number of transducer elements 38. In configurations in which one or more multiplexers 40 and/or 42 are included, a local controller (not shown) responsive to control signals from imaging system 20 provides control signals and configures the multiplexers. Control can be provided algorithmically, or it can be stored in a memory (not shown) within probe handle 14. In some configurations, imaging system 20 is configured to load this memory.

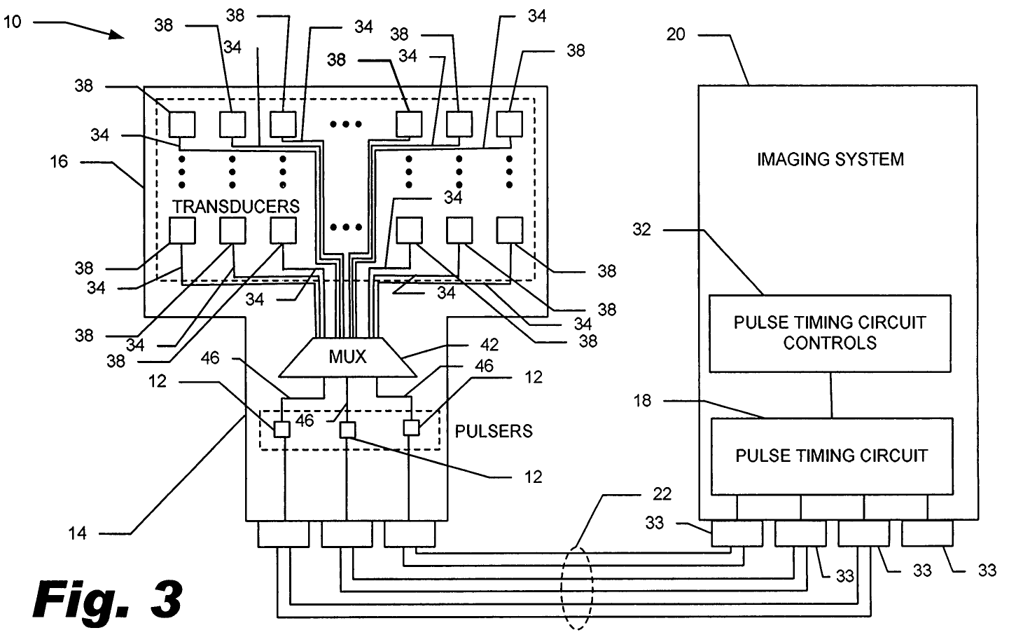

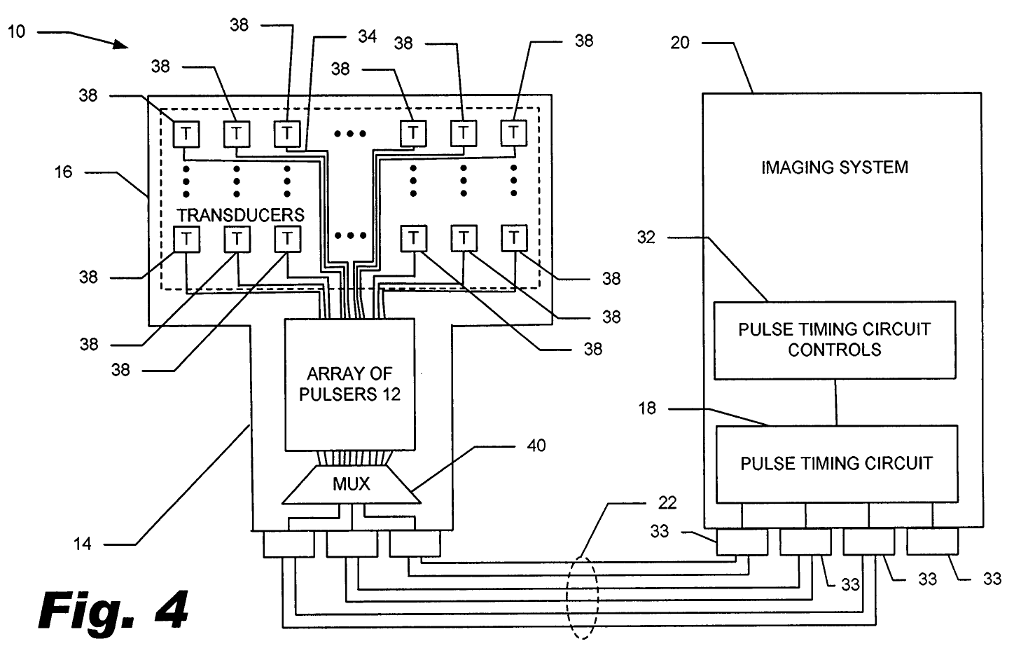

In some configurations and referring to FIG. 3, multiplexing is accomplished by coupling low voltage timing signals directly to individual HV pulse transmitters 12. Outputs of transmitters 12 are followed by an HV multiplexer 42 that maps transmit channels 46 to respective transducer elements 38 for a specified transmit configuration. In various other configurations and referring to FIG. 4, a low voltage multiplexer 40 is used to route low voltage timing signals to a plurality of HV pulse transmitters 12. Each transducer element 38 in these configurations has a dedicated low current HV pulse transmitter 12. Because a low voltage multiplexer 40 is used, some of these configurations can operate on digital data or on analog data, depending on the architecture of imaging system 20.

In some configurations not shown in the Figures, timing circuitry 18 is integrated with HV pulse transmitters 12 in probe handle 14 rather than incorporated into imaging system 20. Imaging system 20 can still be used to generate global timing information such as a start of line pulse or a start of frame pulse, or it can communicate with probe handle 14 via one or more cables 22 to request a series of frames and allow timing circuitry 18 in probe handle 14 to generate frame synchronization. Some of these configurations utilize analog timing information, and others utilize digital timing information. In configurations in which digital timing information is used, one or more digital to analog converters (DACs) such as DACs 24 (illustrated in FIG. 1) located in probe handle 14 are responsively coupled to outputs of co-located timing circuitry 18 to convert the digital timing signals to analog timing signals. The converted analog timing signals are used to drive HV pulse transmitter circuits 12. In configurations utilizing analog timing signals, signal conditioners 28 and/or amplifiers 48 (illustrated in FIG. 2) convert the low voltage analog timing signals into driving signals that control HV pulse transmitter circuits 12. In either analog or digital configurations, unipolar, bipolar, and/or multilevel pulses of varying time delay, pulse width, and/or pulse number can be specified by timing (or control) signals generated by imaging system 18 and communicated to handle 14.

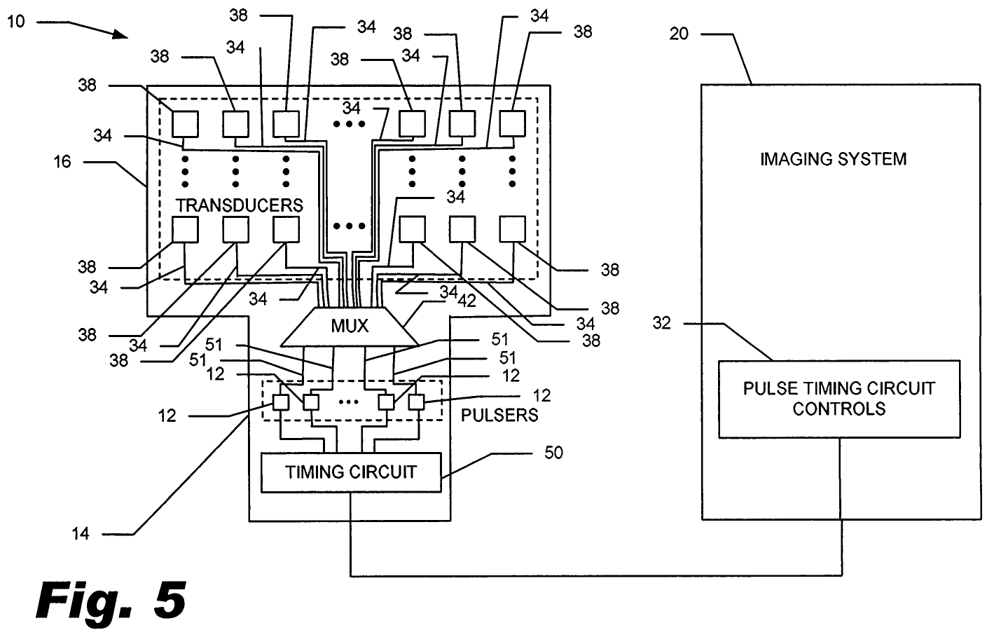

In some configurations and referring to FIG. 5, multiplexing is accomplished by generating low voltage timing signals using a dedicated circuit 50. The low voltage timing signals are coupled directly to individual HV pulse transmitters 12. Outputs of HV pulse transmitters 12 are coupled to an HV multiplexer that maps transmit channels 51 to their respective transducer elements 38 for a selected transmit configuration.

In some configurations, a low voltage multiplexer (not shown in FIG. 5) is provided between timing circuit 50 and pulsers 12, and high voltage multiplexer 42 is omitted. Additional pulsers 12 are provided in some of the configurations to compensate for the omission of multiplexer 42.

In some other configurations similar to that shown in FIG. 4, a low voltage multiplexer 40 routes low voltage timing signals to a plurality of HV pulse transmitters 12 in an array, and each transducer element 38 is associated with its own low current HV pulse transmitter 12. These configurations can be made to operate using either analog data or digital data, depending upon the architecture of imaging apparatus 20.

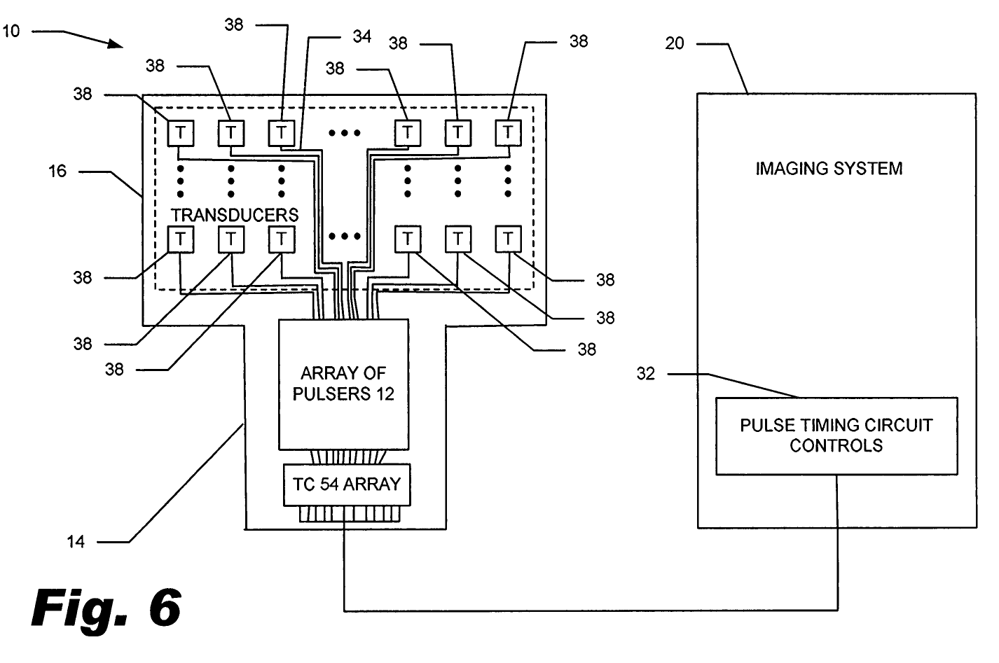

In yet other configurations of the present invention and referring to FIG. 6, each transducer element 38 is associated with its own dedicated high voltage pulser 12 in an array of pulsers. Each high voltage pulser 12 is responsive to a corresponding dedicated reprogrammable timing circuit (TC) 54 in an array of timing circuits. These configurations do not require a multiplexer in circuit as described above. Instead, a single start of frame or start of line signal is propagated in parallel to all timing circuits 54 in the array. In this manner, timing variations between different channels 34 are significantly reduced and phase alignment between channels 34 is greatly improved.

In some configurations, timing circuit 54 comprises a local RAM that stores a description of pulse trains that are used during imaging. Imaging system 20 selects which of these various pulse trains to use to produce an image. In some configurations, timing circuit 54 comprises a parameterized state machine that is configured to accept programs to produce various pulse train waveforms with different pulse durations, number and levels as required.

Although the various configurations described above have components described as being within probe handle 14, the invention does not require that these components be located within this particular portion of probe 16. Some configurations have one or more of these components located elsewhere within probe 16. In general, any component described in the configurations presented in detail herein as being within probe handle 14 can instead be located anywhere within probe 16, not just handle 14. For example, in some configurations, pulsers 12 are integrated with transducers 38 in a location of probe 16 other than handle 14.

Also, the configurations described in detail herein receive signals from a pulse timing and control circuit 32 located in an external system 20. In some configurations of the present invention, pulse timing and control circuit 32 is integrated into probe 16 itself. In some such configurations, there are no control signals sent from external system 20 to probe 16, but some control signals are sent from pulse timing and control circuit 32 back to external system 20.

It will thus be observed that configurations of the present invention provide the ability to transmit with very small elements and with larger numbers of elements than the number of available system channels. Also, configurations of the present invention provide these advantages without the need to provide large numbers of cables between an imaging system and a probe handle, and without presenting an excessively large capacitive load between transducers and pulsers. Moreover, these benefits accrue without the need for excessive power that would otherwise be required of other portable probe configurations.

Although the configurations described herein relate to ultrasonic probes and imaging systems, various configurations of the present invention are applicable to other types of probes having large numbers of transducer elements activated by pulsers, whether or not the probe is used in conjunction with any type of system. Moreover, configurations of the present invention are not limited to imaging systems or probes used in conjunction therewith, but are more broadly applicable to other types of systems 20, which may be referred to more generally as “external systems” herein.

While the invention has been described in terms of various specific embodiments, those skilled in the art will recognize that the invention can be practiced with modification within the spirit and scope of the claims.

1. A probe comprising:
a plurality of transducers; and
a plurality of pulsers within said probe responsive to one or more transmit timing signals received from an external system to transmit pulses to said plurality of transducers.

2. A probe in accordance with claim 1 wherein said plurality of pulsers are responsive to a low voltage analog transmit timing signal.

3. A probe in accordance with claim 1 further comprising a plurality of high voltage multiplexers configured to route said pluses from said pulsers to said plurality of transducers.

4. A probe in accordance with claim 1 further comprising a low voltage multiplexer configured to couple said transmit timing signals received from said external system to said pulsers.

5. A probe in accordance with claim 4 wherein each said transducer is responsive to a dedicated said pulser.

6. A probe in accordance with claim 1 wherein said pulsers comprise pulsers selected from the set consisting of bipolar pulsers, unipolar pulsars, and combinations thereof, and further comprising conversion circuitry configured to convert said transmit timing signals to low voltage signals to operate said pulsers.

7. A probe in accordance with claim 1 further comprising a digital to analog converter (DAC) in said handle, said DAC responsive to a digital transmit timing signal received from the external system to convert the digital transmit timing signal to an analog timing signal, and said pulsers are responsive to said analog timing signal.

8. A probe in accordance with claim 1 wherein said transducers are ultrasound transducers and the pulsers are responsive to one or more transmit timing signals received from an imaging system.

9. A probe comprising:
a plurality of transducers;
a transmit timing circuit within said probe responsive to one or more control signals received from an external system to generate timing signals;
a plurality of pulsers within said probe responsive to said timing signals to generate high voltage pulses; and
a plurality of transducers within said probe responsive to said high voltage pulses.

10. A probe in accordance with claim 9 further comprising a multiplexer configured to selectively couple said high voltage pulses to said transducers.

11. A probe in accordance with claim 9 further comprising a low voltage multiplexer configured to selectively couple said timing signals to said pulsers.

12. A probe in accordance with claim 9 wherein said transducers are ultrasound transducers and said external system is an imaging system.

13. A probe comprising:
a plurality of transducers;
an array of pulsers, each transducer responsive to pulses from a dedicated said pulser;
a low voltage multiplexer responsive to a control signal from an external system and configured to distribute signals to said array of pulsers;
wherein said pulsers are responsive to said signals from said multiplexer to generate pulses to said transducers.

14. A probe in accordance with claim 13 wherein said transducers are ultrasonic transducers and the external system is an imaging system.

15. A probe comprising:
a plurality of transducers;
an array of pulsers, each transducer responsive to pulses from a dedicated said pulser;
an array of transmit timing circuits within said probe responsive to one or more control signals received from an external system to generate timing signals, wherein said timing circuits include a memory;
wherein said pulsers are responsive to said timing signals from said array of timing circuits to generate pulses to said transducers.

16. A probe in accordance with claim 15 wherein a waveform description is stored in said memory.

17. A probe in accordance with claim 15 wherein the waveform description is stored parametrically.

18. A probe in accordance with claim 15 wherein said transducers are ultrasonic transducers and the external system is an imaging system.

19. A probe comprising:
a plurality of transducers;
a plurality of pulsers within said probe responsive to one or more transmit timing signals to transmit pulses to said plurality of transducers;
a transmit timing circuit within said probe configured to generate the one or more transmit timing signals; and
a pulse timing and control circuit configured to control the transmit timing circuit.

20. A probe in accordance with claim 19 configured to send control signals from the pulse timing and control circuit to an external system.

21. A method for operating a transducer probe comprising:
generating one or more signals in an external system;
controlling a plurality of pulsers in a probe utilizing the one or more signals from the external system; and
operating a plurality of transducers utilizing signals from said plurality of pulsers.

22. A method in accordance with claim 21 wherein said signals from the external system comprise timing signals.

23. A method in accordance with claim 22 wherein said operating a plurality of transducers utilizing signals from said plurality of pulsers comprises operating each said transducer utilizing a signal from a dedicated said pulser.

24. A method in accordance with claim 21 further comprising generating timing signals in a handle of the probe utilizing said one or more signals from the external system.

25. A method in accordance with claim 21 wherein the external system is an imaging system and said transducers are ultrasound transducers.

26. A method for operating a transducer probe comprising:
generating one or more signals in the transducer probe;
controlling a plurality of pulsers in the probe utilizing the one or more signals generated in the transducer probe; and
operating a plurality of transducers utilizing signals from said plurality of pulsers.

27. A method in accordance with claim 26 further comprising sending control signals from the probe to an external system.
